(19) United States
(12) Patent Application Publication
(10) Pub. No.: US 2011/0045052 A1
(43) Pub. Date: Feb. 24, 2011

(54) Bioactive Glass Coatings

(75) Inventors: Robert Graham Hill, Berkshire (GB); Molly Morag Stevens, London (GB); Matthew O'Donnell, London (GB)

Correspondence Address: Pepper Hamilton LLP, 400 Berwyn Park, 899 Cassatt Road, Berwyn, PA, 19312-1183, US

(73) Assignee: IMPERIAL INNOVATIONS LTD., London (GB)

(21) Appl. No.: 12/809,098

(22) PCT Filed: Dec. 19, 2008
(86) PCT No.: PCT/GB08/04196

§ 371 Date: Nov. 5, 2010

(30) Foreign Application Priority Data
Dec. 20, 2007 (GB) 0724896.6

(51) Int. Cl. A61K 9/14; A61K 33/30; A61K 33/08; A61K 33/42; A61K 9/00; A61P 43/00; B05D 3/02; C23C 4/04; C25D 13/02

(52) U.S. Cl. 424/423; 424/641; 424/692; 424/602; 424/400; 424/489; 427/189; 427/446; 204/509

(57) ABSTRACT

The present invention relates to bioactive glass coatings. In particular, the present invention relates to bioactive glass coatings for Ti6Al4V alloys and chrome cobalt alloys, wherein the thermal expansion coefficient of the glass coating is matched to that of the alloy. Such coatings have a particular application in the field of medical prosthetics. The bioactive glass comprises (in mol %) 35-53 SiO2; 2-11 Na20; at least 2% of each of CaO, MgO and K20; 0-15 ZnO; 0-2 B202 and 0-9 P205.

The present invention relates to bioactive glass coatings. In particular, the present invention relates to bioactive glass coatings for Ti6Al4V alloys and chrome cobalt alloys, wherein the thermal expansion coefficient of the glass coating is matched to that of the alloy. Such coatings have a particular application in the field of medical prosthetics.

A biologically active (or bioactive) material is one which, when implanted into living tissue, induces formation of an interfacial bond between the material and the surrounding tissue. More specifically, bioactive glasses are a group of surface-reactive glass-ceramics designed to induce biological activity that results in the formation of a strong bond between the bioactive glass and living tissue such as bone. The bioactivity of bioactive glass is the result of a series of complex physiochemical reactions on the surface of the glass under physiological conditions, which results in precipitation and crystallisation of a carbonated hydroxyapatite (HCA) phase.

The rate of development of the hydroxycarbonated apatite (HCA) layer on the surface of the glass provides an in vitro index of bioactivity. The use of this index is based on studies that have indicated that a minimum rate of hydroxyapatite formation is necessary to achieve bonding with hard tissues. Bioactivity can be effectively examined by using non-biological solutions that mimic the fluid compositions found in relevant implantation sites within the body. Investigations have been performed using a variety of these solutions including Simulated Body Fluid (SBF), as described in Kokubo T, J. Biomed. Mater. Res. 1990; 24; 721-735, and Tris-buffered solution. Tris-buffer is a simple organic buffer solution while SBF is a buffered solution with ion concentrations nearly equal to those of human body plasma. Deposition of an HCA layer on a glass exposed to SBF is a recognised test of bioactivity.

Because of the ability of bioactive glasses to interact with living tissue, including hard tissue and soft connective tissue, they have found use in a number of medical applications, one of which is in providing a coating for medical prostheses, including orthopaedic implants.

Metallic prosthetics (formed of metals or metal alloys such as Titanium, Ti6Al4V and chrome cobalt alloys) are widely used. These have good mechanical properties and are non-toxic, but are not biologically active. Their use can result in formation of dense fibrous tissue around the site of implantation, leading to implant failure. Currently, fixation for most implants, such as prostheses used in hip and knee replacement surgery, is improved by cementing in place with an acrylic bone cement. The use of cements can, however, lead to deterioration of adjacent bone. About 20% of hip replacements use cementless fixation procedures, of which the most common involves the use of plasma sprayed hydroxyapatite coating on the prosthesis. The major problem with cementless fixation is the time required for the bone to grow on to the hydroxyapatite coating.

An alternative technique to promote fixation of a medical prostheses is the provision of a prosthesis with a bioactive coating which has good attachment to the prosthesis material and can stimulate interfacial bond formation with surrounding tissue. Bioactive glasses have been proposed to provide such a coating for prostheses. The higher the bioactivity of the bioactive glass, the quicker the surrounding tissue will form a bond with the bioactive glass, and thereby the prosthesis.

Prosthetics may be formed from ceramic, plastic or metal, however the large majority of prosthetics are formed from Ti6Al4V alloy or chrome cobalt alloys. Early patents suggested that a metal prosthesis could be coated with glass by immersing it in molten glass (U.S. Pat. No. 4,234,972). However, this procedure neglected the importance of matching the thermal expansion coefficient (TEC) of the glass to the metal alloy. If there are large differences between the TEC of a glass coating and the TEC of the prosthesis material, differences in thermal expansion during the coating procedure will give rise to thermal stresses which result in cracking and spalling of the coating, wherein the coating chips, fragments and separates. Thus, without TEC matching, the prosthesis-coating interface will be unreliable.

Such studies also neglected oxidation of the metal alloys and phase changes. In the case of Titanium and its alloys, excessive oxidation results in a thick TiO₂ layer which is brittle and thus, even if the coating bonds to the TiO₂, this TiO₂ layer can spoil away. Oxidation of Titanium and Titanium alloys such as Ti6Al4V results in embrittlement at high temperatures (>960° c.), corresponding to the alpha to beta phase transition.

U.S. Pat. No. 4,613,516 describes the importance of TEC matching when bonding a glass to a metal substrate. The glass is applied to the metal substrate in admixture with a cobaltic, cobaltous, nickel or manganese oxide. Measurement of bioactivity for these glasses is not provided. Indeed, the B₂O₃ added to promote sintering could act to increase the network connectivity (NC) of the glass, and subsequently reduce the degradation and bioactivity of the glass. Furthermore, the inclusion of oxides such as nickel oxide to the glasses, in the amounts disclosed in U.S. Pat. No. 4,613,516, would give rise to a significant release of these species within the body, with a cytotoxic effect.

Other attempts to form bioactive glass coatings on Ti6Al4V alloys have produced TEC-matched, well sintered coatings with good interfacial adhesion, but struggled to produce coatings having these properties in combination with sufficient bioactivity according to accepted definitions. In fact, the addition of hydroxyapatite particles and commercially-produced Bioglass® particles to the surface of the glass coatings was used to improve bioactivity (Gomez-Vega et al. J. Biomed. Mater. Res. 46: 549-559 (1999) and Gomez-Vega et al Biomaterials 21(2):105-111 (2000)).

There are a large number of factors that influence the success of a coating composition. To coat a metal or metal alloy successfully, the coating should be: applied below the alpha to beta phase transition temperature of the alloy; preferably applied at or below 750° C. in order to inhibit oxidation of the alloy at the surface; TEC matched to the alloy; applied below the crystallisation temperature onset (T_{c onset}); and sintered to full density.

The applicants have now determined a further important factor. Namely, in order to be bioactive a glass coating should have a predominantly Q² silicate structure corresponding to a network connectivity (NC) value of 2.0. Network Connectivity (NC) is a measure of the average number of bridging bonds per network forming element in the glass structure. NC determines glass properties such as viscosity, crystallisation rate and degradability. For a silica based glass, at a NC of 2.0 it is thought that linear silicate chains exist of infinite molar mass. As NC falls below 2.0, there is a rapid decrease in molar mass and the length of the silicate chains. At an NC above 2.0, the glass becomes a three dimensional network. SiO₂ forms the amorphous network of the bioactive glass, and compositional factors including the molar percentage of SiO₂ in the glass affects its Network Connectivity (NC).

Glasses which have been produced to be suitable for processing via a sintering route, but failing to realise the importance of network connectivity show less than optimal bioactivity, largely as a result of using too high a silica content (Brink et al, J. Biomed Mater. Res. 37 (1997) 114-121; Brink, J. Biomed. Mater. Res 36 (1997) 109-117 and U.S. Pat. No. 6,054,400).

Thus, for a glass to be bioactive, there is a requirement for a highly disrupted glass of low NC. However, the more disrupted the glass network, the more readily the glass will crystallise, reducing its suitability for sintering. Crystallisation must be avoided since: 1) a glass is in a higher energy state than the equivalent crystalline composition, with the result that a glass is always more reactive and hence more bioactive than the equivalent crystal structure; 2) crystallisation inhibits viscous flow sintering which occurs much more readily than solid state sintering processes; 3) highly disrupted glasses undergo predominantly heterogeneous surface crystal nucleation, where crystallisation originates on the glass particle surface.

As a result, glass compositions designed to prevent crystallisation through the use of a less disrupted network consequently have a higher network connectivity and reduced bioactivity. Similarly, glass compositions with a highly disrupted network, which have a low network connectivity, are prone to crystallisation, which also reduces bioactivity.

There are, unsurprisingly, few glass compositions that meet all the criteria necessary to provide a suitable coating composition. There is therefore a need in the art for glass compositions which can be successfully used to provide coatings for Ti6Al4V alloys and chrome cobalt alloys, wherein TEC matching is achieved, undesired effects such as crystallisation, cracking and spalling are avoided and the glass coatings exhibit bioactivity. It is therefore objective of the invention to produce a glass with a TEC to match that of an alloy, but which can sinter at 750° C. or lower (to prevent crystallisation), and which has a NC value close to 2.0, in order to maintain bioactivity.

The applicants have developed a multi-component glass composition as defined herein, which has physical properties making it suitable for successful use as a coating as well as exhibiting bioactivity.

Therefore, in a first aspect the present invention provides a strontium-free bioactive glass comprising 35 to 53 molar % of SiO₂, 2 to 11 molar % of Na₂O, at least 2 molar % of each of CaO, MgO and K₂O, 0 to 15 molar % ZnO and 0 to 3 molar % P₂O₅, 0 to 2 molar % B₂O₃, wherein the combined molar % of SiO₂, P₂O₅ and B₂O₃ is 40 to 54 molar %.

Preferably, the bioactive glass of the first aspect of the present invention comprises 45 to 50 molar % of SiO₂. Preferably, the bioactive glass comprises 8 to 35 molar % CaO. Preferably, the bioactive glass comprises 3 to 11 molar % K₂O. Preferably, the bioactive glass comprises 1 to 3 molar % P₂O₅. Preferably, the bioactive glass comprises 1 to 15 molar % of ZnO, more preferably 1 to 12 molar %. Preferably, the bioactive glass comprises from 1 to 5 molar % of Li₂O. Preferably, the bioactive glass comprises 0 to 10% CaF₂.

The use of multicomponent glass composition advantageously acts to disorder the glass structure and thus stabilise it against crystallisation. This makes the glasses of the present invention suitable for sintering. A glass has a processing window which is defined as the temperature difference between the glass transition temperature and the onset temperature for crystallisation. The greater the difference between the glass transition temperature (T_{g}) and the extrapolated crystallisation onset temperature (T_{c} onset), the larger the processing window. Preferably, glass compositions suitable for sintering have a processing window of greater than 90° C. Preferably, the glasses of the present invention have a processing window of at least 150° C. The extrapolated value for T_{c onset} has been defined here since Tc reduces with decreasing heating rate and during a sintering hold the heating rate is effectively 0 Kmin⁻¹

Moreover, tailoring the multi-component composition of the glass allows the production of a glass with a Thermal Expansion Coefficient (TEC) matched to that of the alloy it is intended to coat. For example, the incorporation of magnesium ions and optionally also zinc ions influences the TEC of a glass, generally increasing TEC, but decreasing it when substituted for CaO.

Preferably, the bioactive glass comprises 5 to 18 molar % MgO. The inclusion of MgO slightly increases Network Connectivity. A small proportion of Mg goes into the silicate glass network, which inhibits crystallisation and promotes viscous flow sintering. In addition, the Mg opens up the processing window between the glass transition temperature (T_{g}) and the onset temperature of crystallisation (T_{c onset}).

Preferably, a glass of the invention has a Network Connectivity of between 1.8 and 2.5, more preferably between 1.9 and 2.4. This range of Network Connectivity is preferable in order to ensure bioactivity of the glass and is primarily achieved by balancing the molar percentages of SiO₂ and P₂O₅ within the glass composition.

A glass of the invention can be used to coat a medical prosthesis, preferably wherein the prosthesis comprises a Ti6Al4V alloy or a chrome cobalt alloy. The Thermal Expansion Coefficient of Ti6Al4V alloy is typically between 8×10⁻⁶K⁻¹ and 10.6×10⁻⁶K⁻¹. Preferably, a bioactive glass for coating a surface comprising Ti6Al4V alloy should have a TEC of 8.8×10⁻⁶K⁻¹ and 12×10⁻⁶K⁻¹. The TEC of the bioactive glass is preferably higher than that of the alloy it is being used to coat, in order to put the glass into compression. Some dissolution of oxides from the surface of the metal alloy into the glass coating will occur and will slightly reduce the TEC of the glass at the interface between the glass and the metal alloy.

The TEC of Chrome Cobalt alloy is typically 12.5×10⁻⁶K⁻¹. Preferably, a bioactive glass for coating a surface comprising Chrome Cobalt alloy should have a TEC of between 11×10⁻⁶K⁻¹ and 14×10⁻⁶K⁻¹, preferably between 12×10⁻⁶K⁻¹ and 14×10⁻⁶K⁻¹. As described above, the TEC of the bioactive glass is preferably higher than that of the alloy it is being used to coat. These preferred TEC ranges are suitable for any Chrome Cobalt alloy, and the bioactive glass coatings of the present invention can be used to coat Chrome Cobalt alloys other than that described in Table 5. In practice, the TECs of Chrome Cobalt alloys differ from one another by less than 1×10⁻⁶K⁻¹.

In a first embodiment of the first aspect of the present invention, the combined molar percentage of Na₂O and K₂O is less than 15 molar % and the bioactive glass has a TEC of between 8.8×10⁻⁶K⁻¹ and 12×10⁻⁶K⁻¹. This glass composition is particularly useful for coating a Ti6Al4V alloy. Preferably, the glass comprises less than 50 molar % SiO₂, at least 2 molar % of MgO and preferably at least 1 molar % of ZnO, and preferably the glass has a Network Connectivity of between 1.9 and 2.4, preferably between 2.1 and 2.4 Preferably, the combined molar % of CaO and MgO does not exceed 40%, preferably the combined molar % of CaO and MgO is from 30-40%, more preferably from 33.27-39.87%. In certain embodiments CaF is absent. In other embodiments where CaF is present, the combined molar % of CaO, MgO and CaF is from 30-40%, more preferably from 33.27-39.87%

Preferably, the bioactive glass of the first embodiment of the first aspect of the present invention comprises 45 to 50 molar % of SiO₂, 1 to 2 molar % P₂O₅, 15 to 35 molar % CaO, 3 to 7 molar % Na₂O, 3 to 7 molar % K₂O, 2 to 4% ZnO, 5 to 18 molar % MgO and 0 to 10 molar % CaF₂. More preferably, the bioactive glass of this embodiment comprises 49 to 50 molar % of SiO₂, 1 to 1.5 molar % P₂O₅, 17 to 33 molar % CaO, 3.3 to 6.6 molar % Na₂O, 3.3 to 6.6 molar % K₂O, 2 to 4 molar % ZnO, 7 to 17 molar % MgO and 0 to 6 molar % CaF₂. Most preferably, the bioactive glass of this first embodiment comprises 49.46 molar % of SiO₂, 1.07 molar % P₂O₅ and 3 molar % ZnO.

In a second embodiment of the first aspect of the present invention, the combined molar percentage of Na₂O and K₂O is less than 30 molar % and the glass has a Thermal Expansion Coefficient of between 11×10⁻⁶K⁻¹ and 14×10⁻⁶K⁻¹, preferably between 12×10⁻⁶K⁻¹ and 14×10⁻⁶K⁻¹. This glass composition is particularly useful for coating a chrome cobalt alloy. Preferably, the bioactive glass comprises less than 52 molar % SiO₂, at least 2 molar % of MgO or at least 1 molar % of ZnO, and has a Network Connectivity of between 1.8 and 2.5. Preferably, the glass comprises a combined molar percentage of Na₂O and K₂O of 15-18 molar %.

Preferably, the bioactive glass of the second embodiment of the first aspect of the present invention comprises 45 to 50 molar % of SiO₂, 1 to 3 molar % P₂O₅, 0 to 2 molar % B₂O₃, 8 to 25 molar % CaO, 7 to 11 molar % Na₂O, 7 to 11 molar % K₂O, 2 to 12% ZnO, 8 to 12 molar % MgO and 0 to 5 molar % CaF₂.

Preferably, the bioactive glass of the present invention is provided in the form of a powder, wherein said powder has a mean particle size of less than 100 μm. Preferably, the glass powder has a mean particle size of less than 50 μm, preferably less than 40 μm, and more preferably less than 10 μm.

The particle size specified above may be achieved by Ball Milling or Vibratory Milling with a Gyro Mill (Vibratory Puck Mill) followed by sieving or, for large quantities of >10 Kg glass, by Jet Milling followed by air classification (essentially centrifugation). Particle size can be determined using Laser Light Scattering or Coulter Counting, preferably Laser Light Scattering.

In certain embodiments, the glasses of the present invention consist essentially of the oxide components recited in the various embodiments described above.

Aluminium is a neurotoxin and inhibitor of in vivo bone mineralization even at very low levels, for example <1 ppm. Therefore, preferably, the glass of the present invention is aluminium-free.

Preferably, the glass is free of iron-based oxides, such as iron III oxides, e.g. Fe₂O₃, and iron II oxides, e.g. FeO.

The glasses of the present invention have been designed specifically with regard to promoting sintering without crystallisation occurring. Thus, the glasses of the present invention remain amorphous on sintering. To achieve this, the compositions are multi-component in nature in order to increase the entropy of mixing and to avoid the stoichiometry of known crystal phases. Ensuring that the NC is fixed at a value of approximately 2 (between 1.8 and 2.5, preferably between 1.9 and 2.4), and designing the glass compositions so as to avoid crystallisation, ensures that the glasses remain bioactive, whilst allowing the TECs to be matched to those of Ti6Al4V and chrome cobalt alloys.

The second aspect of the present invention provides the bioactive glass of the first aspect of the present invention for use in coating a surface comprising a Ti6Al4V or chrome cobalt alloy. Preferably, the second aspect provides the bioactive glass of the first embodiment of the first aspect of the present invention for coating a surface comprising a Ti6Al4V alloy. Preferably, the second aspect also provides the bioactive glass of the second embodiment of the first aspect of the present invention for coating a surface comprising a chrome cobalt alloy. Preferably, the surface comprising Ti6Al4V alloy or Chrome Cobalt alloy is the surface of a prosthesis.

The third aspect of the present invention provides a glass coating comprising the bioactive glass of the first or second aspect of the present invention.

The bioactive glass coating of the present invention may comprise one or more layers of the bioactive glass of the first or second aspect of the present invention. A single layer coating may be provided, as described in Examples 3 and 5. Alternatively, a bilayer coating may be provided. The one or more layers of the coating may all comprise bioactive glass of the first or second aspect of the present invention. Alternatively, the coating may be a bilayer or multi-layer coating in which at least one of the layers comprises a bioactive glass of the first or second aspect of the present invention, and at least one layer does not comprise a bioactive glass of the present invention.

A bilayer coating may comprise two layers of bioactive glass. For example, it may be preferable to provide a less bioactive and more chemically stable base layer and a more bioactive and less chemically stable top layer. Optimum bioactivity is required to promote osseointegration. However, it is also desirable that the alloy remains coated for long time periods in the body. For this reason it is desirable to have a much less reactive base glass layer to ensure that the prosthesis remains coated and a more reactive top coat layer to allow optimum bioactivity. Such coatings can be fabricated by a two step process, as described in Example 4. Both layers may comprise bioactive glasses of the present invention. Alternatively, a bilayer could be provided wherein the base layer comprises a less reactive glass, for example a glass known in the art, and wherein the top layer comprises a bioactive glass of the present invention.

Bilayer coatings may also be provided to prevent dissolution of ions from the prosthesis into the surrounding fluid and/or tissue. Bilayer coatings on chrome cobalt are particularly desirable, since there can be significant dissolution of the oxides of cobalt, nickel and chromium from the protective oxide layer of the alloy into the glass from where they may be released from the glass into the body. For this reason a chemically stable base coating glass composition is preferred. A bilayer coating for use with chrome cobalt alloys therefore preferably comprises a base layer which is chemically stable and non-bioactive, and one or more top layers comprising a bioactive glass according to the present invention. Such bilayer coatings can be fabricated by a two step process, as described in Example 6.

Preferably, the base coating for a chrome cobalt alloy comprises 60-70 mol % SiO₂, 6-23 mol % CaO, 7-13 mol % Na₂O, 3-11 mol % K₂O, 0-5 mol % ZnO and 0-5 mol % MgO. Preferably, the base coating for a Ti6Al4V alloy comprises 60-70 mol % SiO₂, 2-3 mol % P₂O₅, 10-14 mol % CaO, 4-11 mol % Na₂O, 1-7 mol % K₂O and 6-11 mol % MgO.

The coating can be used to coat implants/prostheses for insertion into the body, combining the excellent mechanical strength of implant materials such as Ti6Al4V and chrome cobalt alloys, and the biocompatibility of the bioactive glass. The bioactive glass coating can be applied to the metal implant surface by methods including but not limited to enameling or glazing, flame spraying, plasma spraying, rapid immersion in molten glass, dipping into a slurry of glass particles in a solvent with a polymer binder, or electrophoretic deposition. For example, prosthetics comprising the metal alloy Ti6Al4V can be coated with a bioactive glass by plasma spraying, with or without the application of a bond coat layer.

The bioactive coating allows the formation of a hydroxycarbonated apatite layer on the surface of the prosthesis, which can support bone ingrowth and osseointegration. This allows the formation of an interfacial bond between the surface of the implant and the adjoining tissue. The prosthesis is preferably provided to replace a bone or joint such as comprise hip, jaw, shoulder, elbow or knee prostheses. The prostheses can be for use in joint replacement surgery. The bioactive coating can also be used to coat orthopaedic devices such as the femoral component of total hip arthroplasties or bone screws or nails in fracture fixation devices or dental implants.

The fourth aspect of the present invention provides a prosthesis comprising a Ti6Al4V or chrome cobalt alloy, wherein the prosthesis is coated by a coating comprising a bioactive glass of the first or second aspect of the present invention or a glass coating of the third aspect of the present invention. When the prosthesis comprises Ti6Al4V alloy, the coating preferably comprises a bioactive glass according to the first embodiment of the first aspect of the present invention. When the prosthesis comprises a chrome cobalt alloy, the coating preferably comprises a bioactive glass according to the second embodiment of the first aspect of the present invention. The prosthesis may be, for example, an orthopaedic device/implant, a bone screw or nail or a dental implant.

The fifth aspect of the present invention provides a glass powder comprising the bioactive glass of the first or the second aspect of the present invention, wherein said powder has a mean particle size of less than 100 μm and exhibits a processing temperature window of at least 90° C. Preferably, the glass powder has a mean particle size of less than 50 μm, preferably less than 40 μm, and more preferably less than 10 μm.

The sixth aspect of the present invention provides a method of manufacturing a glass coating on a substrate comprising Ti6Al4V or chrome cobalt alloy, comprising applying a glass of the first or second aspect of the invention preferably in the form of the glass powder of the fifth aspect of the present invention onto the substrate to be coated, and sintering.

Preferably the glass powder is sintered at a temperature of between 600 and 1000° C. Preferably the glass powder is sintered at a temperature below the onset temperature for crystallisation T_{c onset} but at least 50° C. above the glass transition temperature, (Tg) more preferably at least 100° C. above Tg.

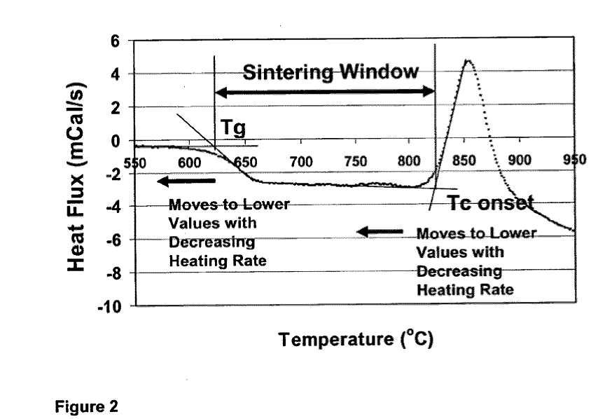

The processing window of a glass is defined as the temperature difference between Tg and the onset temperature for crystallisation as determined by either differential scanning calorimetry (DSC) or differential thermal analysis (DTA), where the glass transition temperature is treated as a quasi-second order thermodynamic transition for the purposes of measurement (see FIG. 2). As described above, the onset temperature of crystallisation is determined by either differential scanning calorimetry (DSC) or differential thermal analysis (DTA). The optimum sintering temperature can be obtained by performing Differential Scanning calorimetry (DSC) over a range of heating rates and extrapolating T_{c onset} to zero heating rate. The greater the temperature difference between Tg and the extrapolated T_{c onset}, the larger the processing window. In general, glass compositions suitable for sintering have a processing window of greater than 90° C.

In a first embodiment of the sixth aspect of the present invention, the glass powder of the fifth aspect of the present invention is deposited onto a surface comprising Ti6Al4V and heated at a rate of between 1 and 60° Cmin⁻¹ to a sintering temperature of between 600° C. and 960° C., below the alpha to beta phase transition temperature.

In a second embodiment of the sixth aspect of the present invention, the glass powder of the fifth aspect of the present invention is deposited onto a surface comprising chrome cobalt alloy and heated to a sintering temperature of between 600° C. and 760° C.

The glass powder of the fifth aspect of the present invention is preferably applied to the substrate to be coated by dip coating in a suspension of glass particles, flame spraying, plasma spraying or electrophoretic deposition. The method of the sixth aspect of the present invention may further comprise the application of cobaltic oxide and/or cobaltous oxide to the surface to be coated, wherein the coating is sintered at a temperature of at least 730° C. Preferably, said cobaltic oxide and/or cobaltous oxide is applied in a total amount of 0.2 and 3.0 weight % of the powdered bioactive glass.

All preferred features of each of the aspect of the invention apply to all other aspects mutatis mutandis.

The invention may be put into practice in various ways and a number of specific embodiments will be described by way of example to illustrate the invention with reference to the accompanying examples and figures, in which:

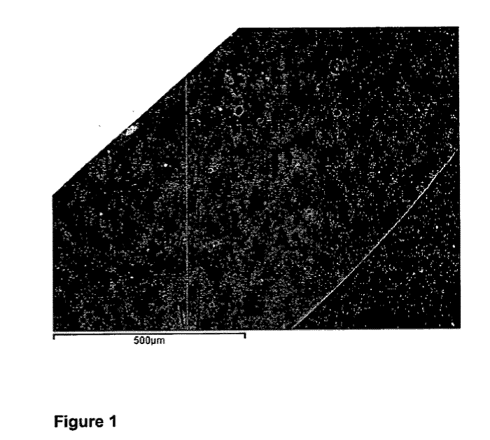

FIG. 1 shows a Scanning Electron Microscopy (SEM) image of a base coat comprising Glass Composition ‘Example 22’ from Table 4 on a Chrome Cobalt Alloy. The composition of this alloy is disclosed in Table 5. This shows the excellent adaptation of the coating to the allow surface and the well sintered coating with relatively little porosity.

FIG. 2 shows a schematic representation of the Sintering or Processing window. As represented by the arrows on this figure, Tg and T_{c onset} move to lower values with decreasing heating rate.

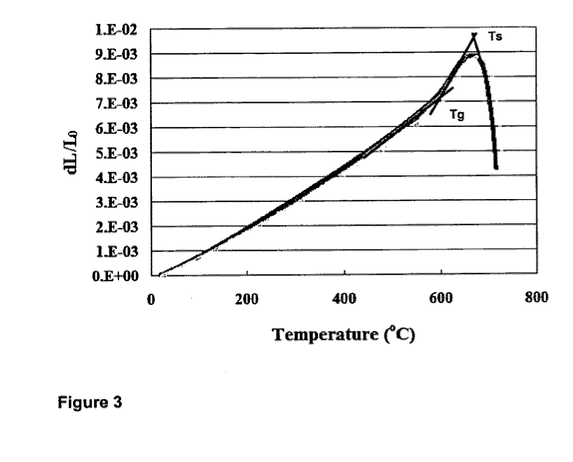

FIG. 3 shows a Dilatometry Curve for Glass 1 from Table 1, showing the glass transition temperature (Tg) and Dilatometric Softening Point (Ts)

The glasses of the present invention are referred to as bioactive glasses. A bioactive glass is one which, when implanted into living tissue, can induce formation of an interfacial bond between the material and the surrounding living tissue. The rate of development of a hydroxycarbonated apatite (HCA) layer on the surface of glass exposed to simulated body fluid (SBF) provides an in vitro index of bioactivity. In the context of the present invention, a glass is considered to be bioactive if, on exposure to SBF for example in accordance with the procedure set out in the following example 1, deposition of a crystalline HCA layer occurs, as can be measured by, for example, Fourier Transform Infra Red Spectroscopy (FTIR). Deposition of an HCA layer representative of bioactivity can be considered to occur if, on exposure to SBF, deposition of a crystalline HCA layer occurs within 7 days, as measured by Fourier Transform Infra Red Spectroscopy (FTIR). More preferably, deposition occurs within three days and more preferably within 24 hours. Alternatively, HCA deposition can be detected using X-ray Powder Diffraction (XRD).

The Thermal Expansion Coefficients of the bioactive glasses were calculated using the method described in Example 7. The Network Connectivity was calculated using the method as described in Example 2.

As is well recognised in the art, glass compositions are defined in terms of the proportions of their oxide components. Preferred glass compositions of the invention are set out in Tables 1 and 2 below. Again, as is recognised in the art the glasses of the present invention can be produced from the oxides making up the glass composition and/or from other compounds that decompose with heat to form the oxides, for example carbonates. The glasses can be produced by conventional melt techniques well known in the art. Melt-derived glass is preferably prepared by mixing and blending grains of the appropriate carbonates or oxides, melting and homogenising the mixture at temperatures of approximately 1250° C. to 1500° C. The mixture is then cooled, preferably by pouring the molten mixture into a suitable liquid such as deionised water, to produce a glass frit which can be dried, milled and sieved to form a glass powder. Sieving can allow a glass powder having a maximum particle size (largest particle dimension) to be obtained. For example, as in the examples set out below a 38 micron sieve can be used to produce a glass powder having a maximum particle size of <38 microns.

### EXAMPLE 1

### Measurement of Bioactivity

### Preparation of Tris-Buffer Solution

For the making of tris-hydroxy methyl amino methane buffer, a standard preparation procedure was taken from USBiomaterials Corporation (SOP-006). 7.545 g of THAM is transferred into a graduated flask filled with approximately 400 ml of deionised water. Once the THAM dissolved, 22.1 ml of 2N HCl is added to the flask, which is then made up to 1000 ml with deionised water and adjusted to pH 7.25 at 37° C.

### Preparation of Simulated Body Fluid (SBF)

The preparation of SBF was carried out according to the method of Kokubo, T., et al., J. Biomed. Mater. Res., 1990. 24: p. 721-734.

The reagents shown in Table A were added, in order, to deionised water, to make 1 litre of SBF. All the reagents were dissolved in 700 ml of deionised water and warmed to a temperature of 37° C. The pH was measured and HCl was added to give a pH of 7.25 and the volume made up to 1000 ml with deionised water.

TABLE A
Reagents for the preparation of SBF

| Order | Reagents | Amount | |
|---|---|---|---|
| 1 | NaCl | 7.996 | g |
| 2 | NaHCO₃ | 0.350 | g |
| 3 | KCl | 0.224 | g |
| 4 | K₂HPO₄•3H₂O | 0.228 | g |
| 5 | MgCl₂•6H₂O | 0.305 | g |
| 6 | 1N HCL | 35 | ml |
| 7 | CaCl₂•2H₂O | 0.368 | g |
| 8 | Na₂SO4 | 0.071 | g |
| 9 | (CH₂OH)CNH₂ | 6.057 | g |

### Powder Assay to Determine Bioactivity:

Glass powder having a particle size of less than 38 microns (achieved by passing through a 38 micron sieve) was added to 50 ml of Tris-Buffer solution or SBF and shaken at 37° C. At a series of time intervals, a sample was removed and the concentration of ionic species was determined using Inductively Coupled Plasma Emission Spectroscopy according to known methods (e.g. Kokubo 1990).

In addition, the surface of the glass is monitored for the formation of an HCA layer by X-ray powder diffraction and Fourier Transform Infra Red Spectroscopy (FTIR). The appearance of hydroxycarbonated apatite peaks, characteristically at two theta values of 25.9, 32.0, 32.3, 33.2, 39.4 and 46.9 in an X-ray diffraction pattern is indicative of formation of a HCA layer. These values will be shifted to some extent due to carbonate substitution and Sr substitution in the lattice. The appearance of a P-O bend signal at a wavelength of 566 and 598 cm⁻¹ in an FTIR spectra is indicative of deposition of an HCA layer.

### EXAMPLE 2

### Calculation of Network Connectivity

Network connectivity (NC) can be calculated according to the method set out in Hill, J. Mater. Sci. Letts., 15, 1122-1125 (1996), but with the assumption that the phosphorus is considered to exist as a separate orthophosphate phase and is not part of the glass network. This assumption is made on the basis of experimental observations of the role of phosphorus in the glass network, including Solid State NMR data.

The NC is calculated as follows:

NC=((4*[SIO₂])−(2*(Σ[Network Modifying Oxide Content]−(3*[P₂O₅]))/[SiO₂]

In order to perform the NC calculations structural assumptions must be made. This calculation assumes that MgO and ZnO act solely as network modifying oxides and do not act as intermediate oxides. For the case of glasses containing fluorides it is assumed that the fluoride is complexed by the cation of highest charge to size ratio and does not form non-bridging fluorides and thus for example when the fluorine is added at CaF₂ it does not influence the NC. For the case of B₂O₃ it can have several roles in the glass network and as its role can not be ascertained it has been neglected in the calculation of NC.

### EXAMPLE 3

### Single Layer Coating of Ti6Al4V

Table 1 sets out a number of exemplary glass compositions which are particularly suitable for coating Ti6Al4V alloys.

Glass composition 1, taken from Table 1, having a particle size <38 microns with a mean particle size of 5-6 microns was coated on to a TiAl6V alloy hip implant by mixing the glass with chloroform containing 1% poly(methylmethacrylate) of molecular weight 50,000 to 100,000 in a weight ratio of 1:5. The femoral stem of the prosthesis was immersed in the chloroform glass suspension then drawn slowly out and the chloroform evaporated off. The temperature of the prosthesis was then raised by between 2 to 60° C./min⁻¹ up to 750° C., above the glass transition temperature of 614° C. but below the onset temperature of crystallisation of 790° C., where it was held for 30 mins under vacuum before cooling to room temperature.

The coated prosthesis had a glossy bioactive glass coating of between 50 and 300 microns thick over the area which had been immersed. When placed in simulated body fluid the coating deposited a hydroxycarbonated apatite layer in under 7 days.

### EXAMPLE 4

### Bilayer Coating of Ti6Al4V

Suitable base coating compositions for a Ti6Al4V alloy and for use in conjunction with a coating layer comprising a glass of the invention are shown in Table 3.

Glass composition 16, taken from Table 3, having a particle size <38 microns with a mean particle size of 5-6 microns was coated on to a TiAl6V alloy hip implant by mixing the glass with chloroform containing 1% poly(methylmethacrylate) of molecular weight 50,000 to 100,000 in a weight ratio of 1:5. The femoral stem of the prosthesis was immersed in the chloroform glass suspension, drawn slowly out and the chloroform evaporated off. The temperature of the prosthesis was then raised by between at 60° C./min⁻¹ up to 450° C. held for 30 mins then raised to 750° C. where it was held for 30 mins under vacuum before cooling to room temperature.

The process was repeated with glass composition 2, taken from Table 1. The coated prosthesis had a glossy bioactive glass coating of between 50 and 300 microns thick over the area which had been immersed.

### EXAMPLE 5

### Single Layer Coating for Chrome Cobalt Alloy

Table 2 sets out a number of exemplary glass compositions which are particularly suitable for coating a chrome cobalt alloy (for example having the composition shown in Table 5).

Glass composition 15, taken from Table 2, having a particle size <38 microns with a mean particle size of 5-6 microns was coated on to a Chrome Cobalt alloy hip implant by mixing the glass with chloroform containing 1% poly(methylmethacrylate) of molecular weight 50,000 to 100,000 in a weight ratio of 1:5. The femoral stem of the prosthesis was immersed in the chloroform glass suspension, drawn slowly out and the chloroform evaporated off.

The temperature of the prosthesis was then raised by between 2 to 60° C./min⁻¹ to 450° C. held for 10 mins then ramped up to 800° C. where it was held for 30 mins under vacuum before cooling to room temperature.

As can be seen from glass example 8 in Table 2, a strontium-free glass composition having 35 to 53 molar % (preferably 45 to 50%) SiO₂, 2 to 11 molar % Na₂O, at least 2 molar % of each of CaO, MgO and K₂O, 0 to 15 molar % ZnO, 0 to 2 molar % B₂O₃ and 0 to 9 molar % P₂O₅ was prepared. Preferably, this composition comprises 8 to 10 molar % of each of P₂O₅, CaO, Na₂O, K₂O, ZrO and MgO.

### EXAMPLE 6

### Bilayer Coatings for Chrome Cobalt Alloys

Suitable base coating compositions for a chrome cobalt alloy and for use in conjunction with a coating layer comprising a glass of the invention are shown in Table 4.

Glass composition 22 taken from Table 4, having a particle size <38 microns with a mean particle size of 5-6 microns was coated on to a Chrome Cobalt alloy hip implant by mixing the glass with chloroform containing 1% poly(methylmethacrylate) of molecular weight 50,000 to 100,000 in a weight ratio of 1:5. The femoral stem of the prosthesis was immersed in the chloroform glass suspension, drawn slowly out and the chloroform evaporated off.

The temperature of the prosthesis was then raised by between 2 to 60° C./min⁻¹ to 450° C. held for 10 mins then ramped up to 750° C. where it was held for 30 mins under vacuum before cooling to room temperature.

The process was then repeated with glass composition 15, taken from Table 2 but the final hold temperature was 800° C.

The coated prosthesis had a glossy bioactive glass coating of between 50 and 300 microns thick over the area which had been immersed. When placed in SBF the coating caused deposition of a hydroxycarbonated apatite layer in under 7 days as evidenced by FTIR.

### EXAMPLE 7

### Estimation of Thermal Expansion Coefficients (TEC)

TEC values were calculated using Appen Factors (Cable, M., Classical Glass Technology (Chapter 1), in Glasses and Amorphous Materials, J. Zarzycki, Editor. 1991, VCH: Weinheim). Appen Factors are empirical parameters based on previously studied silicate glasses. The Appen factor calculations were carried out in two ways, the first of which discounting the Appen Factor for phosphate (i.e. Appen factor calculations not including the presence of phosphate) and the second in which the Appen factor for phosphate was used. In the first calculation, it is considered that phosphate is present as orthophosphate and is present as a second nanoscale glass phase dispersed in a silicate glass matrix phase. An assumption is made that that the matrix silicate phase will determine the TEC. In order to perform the calculation, an assumption is made that Ca²⁺ and Na⁺ ions will charge balance the orthophosphate phase in the ratio present in the overall glass composition. The composition of the silicate phase is then recalculated (after allowing for the charge balancing of the orthophosphate phase), and the Appen calculation of the TEC is performed.

The calculation was performed for glass composition 1 of Table 1, the TEC of which was determined to be 10.9×10⁻⁶K⁻¹. Using the second calculation, the TEC of this glass was determined to be 9.69×10⁻⁶K⁻¹.

### EXAMPLE 8

### Determining the Thermal Expansion Coefficient Using Dilatometry

Dilatometry was carried out using a Netzch dilatometer in order to determine the dilatometric softening temperature (Ts) and the thermal expansion coefficient (TEC) for each glass. The 20 mm cast bar samples were analyzed between 30° C. and their glass transition temperatures (identified from DSC analysis) at a rate of 5° C./min. The TEC and Ts were determined from each trace using the system software. In some cases glasses were observed to flow very readily after Ts.

### EXAMPLE 9

### Preparation of Glasses

Exemplary glasses of the present invention are set out in Tables 1 and 2. These glasses can be produced by well-known melt-quench production techniques. Glass 1 was prepared as follows. The same procedure can be followed in order to produce the other glasses of the invention by appropriately varying the proportions of the oxides/carbonates used.

49.49 g of silica in the form of quartz, 2.53 g of phosphorus pentoxide, 54.37 g of calcium carbonate, 5.82 g of sodium carbonate, 7.60 g of potassium carbonate 4.07 g of zinc oxide and 4.87 g of magnesium oxide were mixed together and placed in a platinum crucible and melted at 1440° C. for 1.5 hours then poured into demineralised water to produce a granular glass frit. The frit was dried then ground in a vibratory mill to produce a powder.

### EXAMPLE 10

### DSC Calculation for Glass 1

Differential scanning calorimetry (DSC) analysis was carried out on glass 1 as listed in table 1. The results of this analysis identified a Tg onset of 604° C., a crystallisation onset of 808° C. and, consequently, a processing/sintering window of 204° C. DSC analysis was carried out using a Stanton Redcroft DSC 1500 instrument and, in some cases, a Stanton Redcroft DTA/TGA 1600.

In some cases it can be difficult to determine T_{c onset} accurately, particularly if the crystallisation process is sluggish. In all cases T_{c onset} is above 750° C. Consequently, the processing window for the glasses of the invention can be said to be >(750° C.−Tg). Taking this into account, all glasses shown within table 1 have a processing window of >152° C.

TABLE 1
Bioactive Glass Compositions for Coating Ti6Al4V Alloys in Mole Percent, NC Values, TEC
Values (calculated using the second calculation of example 7) and Dilatometric Tg Values

| | | | | | | | | | | TEC | Tg (Dil) |
|---|---|---|---|---|---|---|---|---|---|---|---|
| Glass | SiO₂ | P₂O₅ | CaO | Na₂O | K₂O | ZnO | MgO | CaF₂ | NC | (×10⁻⁶K⁻¹) | (° C.) |
| 1 | 49.46 | 1.07 | 32.62 | 3.30 | 3.30 | 3.00 | 7.25 | | 2.13 | 9.69 | 591 |
| 2 | 49.46 | 1.07 | 26.02 | 3.30 | 3.30 | 3.00 | 13.85 | | 2.13 | 9.23 | 598 |
| 3 | 49.46 | 1.07 | 20.02 | 3.30 | 3.30 | 3.00 | 13.85 | 6.00 | 2.37 | 9.23 | 575 |
| 4 | 49.46 | 1.07 | 20.02 | 5.30 | 5.30 | 3.00 | 13.85 | | 2.21 | 10.17 | 580 |
| 5 | 49.46 | 1.07 | 17.02 | 6.60 | 6.60 | 3.00 | 16.25 | | 2.13 | 11.04 | 573 |
| 6 | 49.46 | 1.07 | 19.02 | 5.30 | 5.30 | 3.00 | 16.25 | | 2.15 | 10.18 | 580 |

TABLE 2
Bioactive Glass Compositions for Coating Chrome Cobalt Alloys in Mole Percent

| | | | | | | | | | | | TEC |
|---|---|---|---|---|---|---|---|---|---|---|---|
| Glass | SiO₂ | P₂O₅ | B₂O₃ | CaO | Na₂O | K₂O | ZnO | MgO | CaF₂ | NC | (×10⁻⁶ K⁻¹) |
| 7 | 45.00 | 1.62 | — | 11.66 | 7.44 | 10.36 | 11.97 | 11.93 | 0 | 1.84 | 12.52 |
| 8 | 49.09 | 8.42 | — | 8.24 | 8.65 | 8.72 | 8.34 | 8.35 | — | | |
| 9 | 45.00 | 3.00 | — | 20.00 | 10.00 | 8.00 | 4.00 | 10.00 | — | 2.09 | 13.20 |
| 10 | 50.00 | 3.00 | — | 15.0 | 10.00 | 8.00 | 4.00 | 10.00 | — | 2.48 | 12.74 |
| 11 | 49.00 | 3.00 | — | 15.0 | 10.00 | 8.00 | 4.00 | 10.00 | — | 2.45 | 12.70 |
| 12 | 46.00 | 3.00 | — | 23.0 | 8.00 | 7.00 | 3.00 | 10.00 | — | 2.17 | 12.32 |
| 13 | 45.00 | 3.00 | — | 20.0 | 8.00 | 7.00 | 3.00 | 10.00 | 4 | 2.27 | 11.90 |
| 14 | 45.00 | 2.00 | 2.0 | 24.00 | 8.00 | 7.00 | 2.00 | 9.00 | — | 2.04 | 12.17 |
| 15 | 45.00 | 1.62 | 0 | 11.66 | 7.44 | 10.36 | 11.97 | 11.93 | | 1.84 | 12.52 |

The glasses described above were determined by DSC to have Tg values between 540 and 570° C.

TABLE 3
Base Coating Compositions for Ti6Al4V in Mole Percent

| Glass | SiO₂ | P₂O₅ | CaO | Na₂O | K₂O | ZnO | MgO |
|---|---|---|---|---|---|---|---|
| 16 | 61.34 | 2.55 | 13.55 | 10.01 | 1.79 | — | 10.56 |
| 17 | 68.40 | 2.56 | 10.93 | 4.78 | 6.78 | — | 6.57 |
| 18 | 67.40 | 2.56 | 11.93 | 4.78 | 6.78 | — | 6.57 |

TABLE 4
Base Coating Compositions for Chrome Colbalt Alloy in Mole_Percent

| Glass | SiO₂ | CaO | Na₂O | K₂O | ZnO | MgO |
|---|---|---|---|---|---|---|
| 19 | 61.10 | 22.72 | 12.17 | 4.00 | — | — |
| 20 | 66.67 | 6.28 | 7.27 | 10.62 | 4.47 | 4.70 |
| 21 | 68.54 | 14.72 | 9.11 | 7.63 | — | — |
| 22 | 66.67 | 15.56 | 9.29 | 7.24 | 0.23 | — |

TABLE 5
Composition of CoCr alloy used

| | | | Molyb- | | | |
|---|---|---|---|---|---|---|
| | Cobalt | Chromium | denum | Silicon | Manganese | Carbon |
| Element | (Co) | (Cr) | (Mo) | (Si) | (Mn) | (C) |
| Weight % | 64.8 | 28.5 | 5.3 | 0.5 | 0.5 | 0.4 |

1. A strontium-free bioactive glass comprising 35 to 53 molar % of SiO₂, 2 to 11 molar % of Na₂O, at least 2 molar % of each of CaO, MgO and K₂O, 0 to 15 molar % ZnO and 0 to 3 molar % P₂O₅, 0 to 2 molar % B₂O₃, wherein the combined molar % of SiO₂, P₂O₅ and B₂O₃ is 40 to 54 molar %.

2. The bioactive glass of claim 1, comprising 45 to 50 molar % of SiO₂.

3. The bioactive glass of claim 1, comprising 8 to 35 molar % CaO.

4. The bioactive glass of claim 1, comprising 5 to 18 molar % MgO.

5. The bioactive glass of claim 1, comprising 3 to 11 molar % K₂O.

6. The bioactive glass of claim 1, comprising 1 to 3 molar % P₂O₅.

7. The bioactive glass of claim 1, further comprising from 1 to 5 molar % of ZnO.

8. The bioactive glass of claim 1 further comprising from 1 to 5 molar % of Li₂O.

9. The bioactive glass of claim 1, further comprising from 0 to 10% CaF₂.

10. The bioactive glass of claim 1, wherein the combined molar percentage of Na₂O and K₂O is less than 15 molar % and wherein the bioactive glass has a Thermal Expansion Coefficient of between 8.8×10⁻⁶K⁻¹ and 12×10⁻⁶K⁻¹.

11. The bioactive glass of claim 10, wherein the glass comprises less than 50 molar % SiO₂, at least 2 molar % of MgO or at least 1 molar % of ZnO, and wherein the glass has a Network Connectivity of between 1.9 and 2.4.

12. The bioactive glass of claim 1 comprising 45 to 50 molar % of SiO₂, 1 to 2 molar % P₂O₅, 15 to 35 molar % CaO, 3 to 7 molar % Na₂O, 3 to 7 molar % K₂O, 2 to 4 molar % ZnO, 5 to 18 molar % MgO and 0 to 10 molar % CaF₂.

13. The bioactive glass of claim 12 comprising 49 to 50 molar % of SiO₂, 1 to 1.5 molar % P₂O₅, 17 to 33 molar % CaO, 3.3 to 6.6 molar % Na₂O, 3.3 to 6.6 molar % K₂O, 2 to 4 molar % ZnO, 7 to 17 molar % MgO and 0 to 6 molar % CaF₂.

14. The bioactive glass of claim 13, comprising 49.46 molar % of SiO₂, 1.07 molar % P₂O₅ and 3 molar % ZnO.

15. The bioactive glass of claim 1, wherein the combined molar percentage of Na₂O and K₂O is less than 30 molar % and wherein the glass has a Thermal Expansion Coefficient between 11×10⁻⁶K⁻¹ and 14×10⁻⁶K⁻¹.

16. The bioactive glass of claim 15, wherein said bioactive glass comprises less than 52 molar % SiO₂, at least 2 molar % of MgO and at least 1 molar % of ZnO, and has a Network Connectivity of between 1.8 and 2.5.

17. The bioactive glass of claim 1, comprising 45 to 50 molar % of SiO₂, 1 to 3 molar % P₂O₅, 0 to 2 molar % B₂O₃, 8 to 25 molar % CaO, 7 to 11 molar % Na₂O, 7 to 11 molar % K₂O, 2 to 12 molar % ZnO, 8 to 12 molar % MgO and 0 to 5 molar % CaF₂.

18. A strontium-free bioactive glass comprising 35 to 53 molar % SiO₂, 2 to 11 molar % Na₂O, at least 2 molar % of each of CaO, MgO and K₂O, 0 to 15 molar % ZnO, 0 to 2 molar % B₂O₃ and 0 to 9 molar % P₂O₅.

19. The bioactive glass of claim 18 comprising 8 to 10 molar % of each of P₂O₅, CaO, Na₂O, K₂O, ZnO and MgO.

20-23. (canceled)

24. A glass coating comprising the bioactive glass of claim 1.

25. The glass coating of claim 24, wherein the glass coating is a bilayer coating and at least one of the two layers making up the bilayer coating comprises the bioactive glass.

26. A prosthesis comprising a Ti6Al4V or chrome cobalt alloy wherein the prosthesis is coated by a coating comprising a bioactive glass of claim 1.

27. The prosthesis of claim 26, wherein the prosthesis comprises Ti6Al4V and wherein the coating comprises a bioactive glass of claim 10.

28. The prosthesis of claim 24, wherein the prosthesis comprises a chrome cobalt alloy and wherein the coating comprises a bioactive glass of claim 15.

29. A glass powder comprising the bioactive glass of claim 1, wherein said powder has a mean particle size of less than 100 μm and exhibits a processing temperature window of at least 90° C.

30. The glass powder of claim 29 wherein the powder has a mean particle size of less than 50 μm.

31. A method of manufacturing a glass coating on a substrate comprising Ti6Al4V or chrome cobalt alloy, comprising applying the glass powder of claim 29 onto the substrate to be coated and sintering.

32. The method of claim 31 wherein the glass powder is sintered at a temperature of between 600 and 1000° C.

33. The method of claim 32 wherein the glass powder is sintered at a temperature below the onset temperature for crystallisation but at least 50° C. above the glass transition temperature.

34. The method of claim 33 wherein the glass powder is sintered at a temperature at least 100° C. above the glass transition temperature.

35. The method of claim 32 wherein said glass powder is deposited onto a surface comprising Ti6Al4V and heated at a rate of between 1 and 60° Cmin⁻¹ to a sintering temperature of between 600 and 960° C.

36. The method of claim 32 wherein said glass powder is deposited onto a surface comprising chrome cobalt alloy and heated to a sintering temperature of between 600 and 760° C.

37. The method of claim 32 wherein the glass powder is applied to the substrate to be coated by dip coating in a suspension of glass particles, flame spraying, plasma spraying or electrophoretic deposition.

38. The method of claim 32 further comprising the application of cobaltic oxide and/or cobaltous oxide to the surface to be coated, wherein the coating is sintered at a temperature of at least 730° C.

39. The method of claim 38 wherein said cobaltic oxide and/or cobaltous oxide is applied in a total amount of 0.2 and 3.0 weight % of the powdered bioactive glass.

40. (canceled)

41. A prosthesis comprising a Ti6Al4V or chrome cobalt alloy wherein the prosthesis is coated by a coating comprising the glass coating of claim 24.
